(19) United States
(12) Patent Application Publication
(10) Pub. No.: US 2009/0054994 A1
(43) Pub. Date: Feb. 26, 2009

(54) Methods and kits for prophylactically reinforcing degenerated spinal discs and facet joints near a surgically treated spinal section

(76) Inventors: James Rogan, Austin, TX (US); Brian Burkinshaw, Pflugerville, TX (US); Steven I. Whitlock, Austin, TX (US)

Correspondence Address: ANDREWS KURTH LLP, 1350 I STREET, N.W., SUITE 1100, WASHINGTON, DC, 20005, US

(21) Appl. No.: 11/892,218

(22) Filed: Aug. 21, 2007

(51) Int. Cl. A61F 2/02; A61F 2/44

(52) U.S. Cl. 623/23.72; 623/17.11; 623/23.75

(57) ABSTRACT

A method is effective for prophylactically treating discs and facet joints near a spinal section that requires surgery. The method comprises the steps of performing a surgical procedure on a section of a spine, and reinforcing a disc or a facet joint adjacent to the surgically treated area with an effective amount of an injected, in situ curable biocompatible matrix or biocompatible polymeric compound. The procedure delays or prevents the development of permanent or irreversible degenerative changes in the treated disc and/or facet joint, thus offering the spinal surgery patient a higher probability of long-term success for arresting disc and facet joint degeneration and prevention of latent adjacent disc/facet joint problems.

### TECHNICAL FIELD

The technical field relates to a treatment for degenerative disc diseases and, in particular, to a procedure that facilitates the repair of a defective spinal section utilizing injection of in situ curable materials to prophylactically reinforce adjacent spinal discs, facet joints and spinal structures that are compromised or at risk of degeneration.

### BACKGROUND

The spinal column is composed of a series of connected bones called vertebrae. The vertebrae surround the spinal cord and protect it from damage. Nerves branch off the spinal cord and travel to the rest of the body, allowing for communication between the brain and the body.

As shown in FIG. 1, the vertebrae are connected by spongy intervertebral discs and two small joints called facet joints. The intervertebral disc, which is made up of strong connective tissues that hold one vertebra to the next, acts as a cushion or shock absorber between the vertebrae. The disc and facet joints allow for movements of the vertebrae.

An intervertebral disc is composed of a tough outer layer called the “annulus fibrosus” and a gel-like center called “nucleus pulposus” (FIG. 2). The annulus fibrosus is a strong radial tire-like structure made up of lamellae. The lamellae are concentric sheets of collagen fibers connected to the vertebral end plates. The sheets are orientated at various angles. The annulus fibrosus encloses the gel-like nucleus pulposus.

Disc degeneration commonly occurs during aging. As people age, the nucleus pulposus begins to lose water content, making the disc less effective as a cushion. As a disc continues to deteriorate, the annulus fibrosus can eventually tear. These internal disc disruptions (IDD) are known to allow the displacement of the nucleus pulposus through the tear in the annulus fibrosus to the highly innervated outer ⅓ of the annulus and into the spaces occupied by the nerve roots and spinal cord (this is sometimes also called “Leaky Disc Syndrome”). IDD can act as stress concentration sites that severely weaken the structural integrity of the annulus. It is not uncommon for the tears to result, producing a herniated disc.

Another form of disc degeneration is the “herniated disc”. As shown in FIG. 2, a spinal disc, having lost water content and structural integrity, or having been subjected to excessive stresses due to injury, will develop a weakened annulus fibrosus. The areas of the annulus fibrosus subjected to the highest stresses (usually near the posterior aspect of the disc) are most prone to stress injuries manifesting in the forms of tears, or herniation of the annular fiber structures. The herniation can then press on the nerves, spinal cord, and spinal nerve roots found outside the disc and cause pain, numbness, tingling and/or weakness in the extremities.

Treatments for disc degeneration and herniated discs include local injection of anti-inflammatory medications, such as steroids and non-steroid anti-inflammatory drugs (NSAIDs), physical therapy, behavior modification, transcutaneous electrical nerve stimulation (TENS), intradiscal electrothermal therapy (IDET), radio frequency (RF) therapy, and surgery. The surgery can be performed as either a conventional open surgery, a mini-open surgery using very small opening incisions, or percutaneously using specially designed instruments and radiographic techniques. One form of surgery is referred to as discectomy. Typically, all or part of the degenerated or herniated disc tissue is removed to relieve the pressure on the neural structures. In more severe cases, where the disc has completely degenerated and/or is no longer structurally functional, the entire disc is removed, and a vertebral fusion is required. In order to maintain the normal height of the disc space and prevent the vertebrae from collapsing and rubbing together, a bone graft often is placed between the adjacent vertebrae and, in most cases, a small metal plate is implanted to stabilize the spine while it heals. The body heals by incorporating the graft and fusing the bones.

Spinal fusion, however, often causes loss of spinal mobility and increases stress on adjacent discs and facet joints, accelerating degeneration of these discs and joints. Moreover, orthopaedic surgeons have often noted that when performing fusions or open discectomies on a degenerated disc, discs near the degenerated disc appeared to be of marginal health or clearly compromised. For example, the adjacent discs may appear dehydrated, (i.e.: >20% reduced height), be gray or even black on magnetic resonance imaging (MRI) (indicating a degenerated or dying condition), or show other signs of disc degeneration. These compromised discs have a high probability of degenerating further over time, especially in light of the fusion or discectomy being performed on the adjacent disc. There is, however, no known procedure for prophylactically treating these discs to slow, retard or arrest the degeneration process. In these cases, one or more disc(s) adjacent to the most severely degenerated disc may be considered to be in imminent danger of failure. A surgeon may elect to perform a discectomy or fusion on such a disc at the same time in order to avoid a future surgery, simply because there is no other prophylactic treatment to slow, retard or arrest the degeneration of the adjacent disc(s).

Still another form of degeneration to the spine occurs at the facet joints. The facet joints, or joints with “small faces”, are found at every spinal level (except at the top level) and provide about 20% of the torsional (twisting) stability in the neck and low back. Each upper half of the paired facet joints are attached on both sides on the backside of each vertebra, near its side limits, then extend downward. These faces project forward or towards the side. The other halves of the joints arise on the vertebra below, then project upwards, facing backward or towards the midline, to engage the downward faces of the upper facet halves. The facet joints slide on each other and both sliding surfaces are normally coated by a very low friction, moist cartilage. A small sack or capsule surrounds each facet joint and provides a sticky lubricant for the joint. Each sack has a rich supply of tiny nerve fibers that provide a warning when irritated.

Facet joints are in almost constant motion with the spine and commonly wear out or become degenerated as the disc space narrows due to aging and disc dehydration. When facet joints become worn or torn, the cartilage may become thin or disappear resulting in bone-on-bone contact and or boney facet joint abnormalities. The resulting osteoarthritis can produce considerable back pain on motion. This condition may also be referred to as “facet joint disease” or “facet joint syndrome”.

Treatments for facet joint disease include anti-inflammatory medications, muscle relaxants, physical therapy, and facet joint injections. When anti-inflammatory medications, muscle relaxants and physical therapy have not provided relief of your pain, injection of the painful facet joint with a local anesthetic and steroid medication may be necessary. Frequently these injections can provide long-term pain relief. If the pain returns, the facet joints can be injected again. If there is temporary relief and no surgically correctable problem, the nerves which supply sensation to the facet joint can be interrupted. This is done by a procedure called radiofrequency facet nerve lesioning which uses radiofrequency energy. More invasive and less proven surgical therapies include the placement of “spacers” between the spinous processes to maintain joint spacing and relieve pressure on the facets or facet joint implants. Fusion of the entire level is typically the final step in this treatment process.

### SUMMARY

What is disclosed is a method for prophylactically treating discs and facet joints adjacent to or relatively near a spinal section that requires surgery. The method comprises the steps of performing a surgical procedure on a section of a spine; and reinforcing a disc or a facet joint relatively near said section of the spine with an effective amount of a biocompatible matrix or biocompatible polymeric compound.

Also disclosed is a kit for prophylactically treating discs and facet joints relatively near a spinal section that requires surgery. The kit comprises components needed for the formation of a biocompatible matrix or biocompatible polymeric compound; a needle or a catheter for delivering the biocompatible matrix or biocompatible polymeric compound or components thereof into a disc annulus, around the exterior of the disc, and into the facet joints; a delivery device for injecting the biocompatible matrix or biocompatible polymeric compound or components thereof and a spine stabilization device.

### DESCRIPTION OF THE DRAWINGS

The detailed description will refer to the following drawings in which:

FIG. 1 is a drawing of a spinal column.

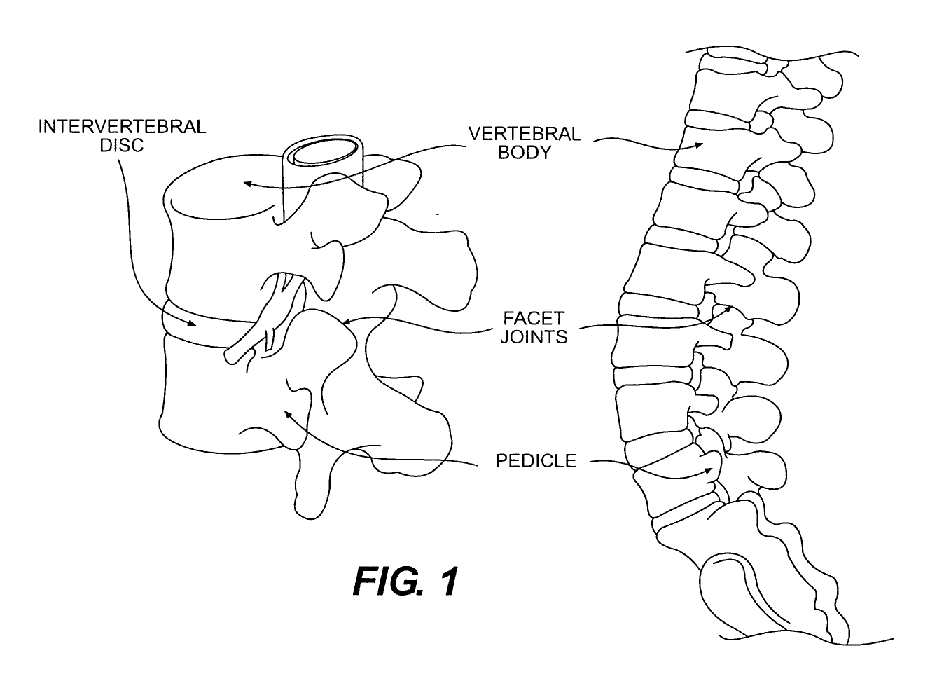

FIG. 2A is a cross-sectional view of a vertebral body at the disk space exhibiting hernia which may be treated according to the herein disclosed embodiments.

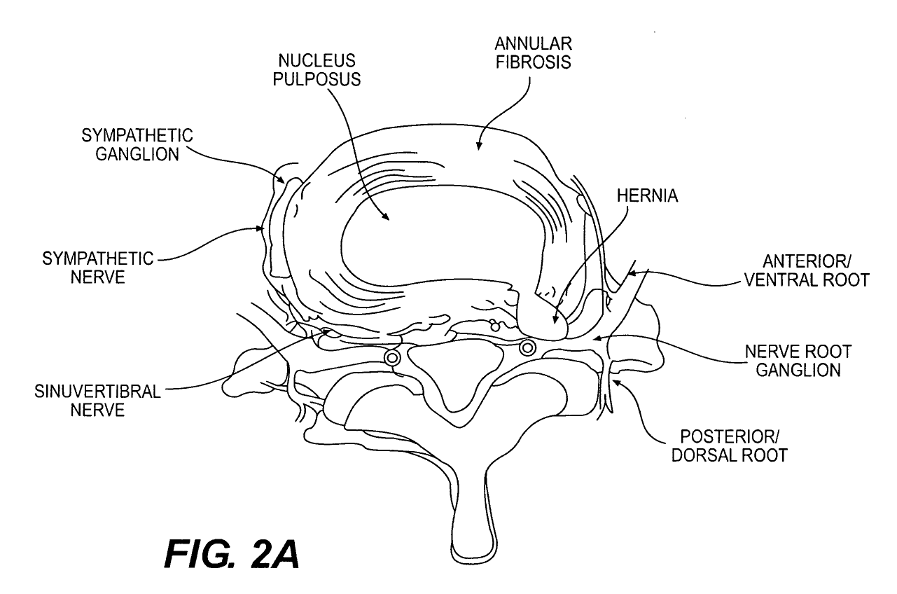

FIG. 2B is a cross-sectional view of a vertebral body at the disk space exhibiting internal disc disruptions, (IDD) or “leaky disc syndrome” which may be treated according to the herein disclosed embodiments.

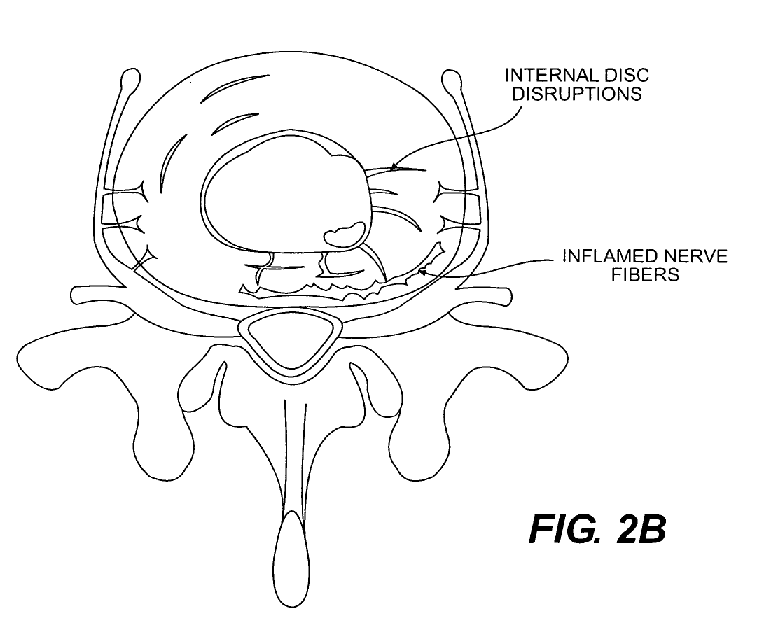

FIG. 3 is a flow chart showing an embodiment of the method for prophylactically treating discs and facet joints near a spinal section that requires surgery.

FIG. 4 illustrates an embodiment of a delivery device for injecting fluids into a spinal disc.

FIGS. 5A and 5B are fluoroscopy x-rays (discography) of a spinal disc before and after treatment.

FIG. 5C is a fluoroscopic x-ray of a facet joint injection.

### DETAILED DESCRIPTION

Discs and facet joints relatively near a degenerated disc or a defective region of spinal column are often abnormal or in compromised health conditions. These discs and facet joints are also subject to increased stress after surgical treatment to the degenerated disc or the defective region of spinal column. A number of devices, such as the Dynesis® system, the Wallis® system, the DIAM™ system, PDN® (Prosthetic Disc Nucleus), NuCore™ Injectable Nucleus, BioDisc™, Aguarelle™, and Newcleus™, have been developed to stabilize a degenerated disc, these devices typically use artificial, synthetic and non-resorbable materials that may interfere with the self-healing process. NuCore™ and BioDisc™ are examples of artificial, synthetic and/or non-resorbable therapies that do not allow natural healing. Moreover, most of these therapies and devices disrupt the disc architecture. For example, the Dynesis® device is mounted on the vertebrae by titanium screws, and implantation of PDN®, Aguarelle™ or Newcleus™ devices creates a significant hole in the disc that rarely can be sealed well enough to prevent leakage of disc nucleus material. Accordingly, it is difficult to justify the use of these invasive devices for the treatment of compromised discs that have not yet led to clinical symptoms.

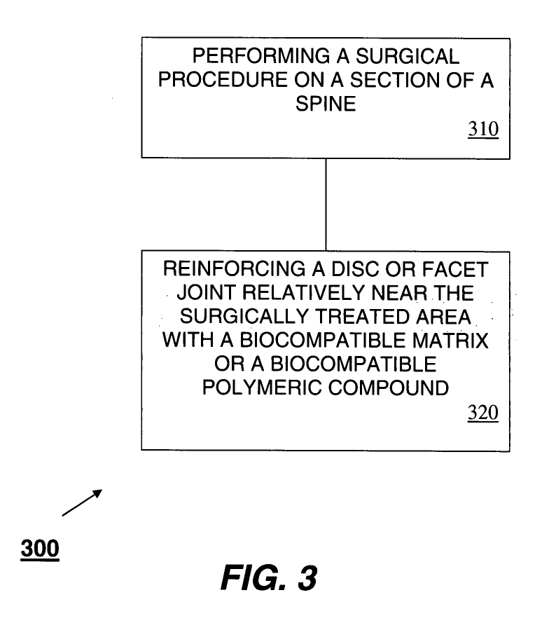

A method is disclosed to prophylactically treat discs and facet joints relatively near a spinal section that requires surgery, via the injection of in situ curable biocompatible matrix or biocompatible polymeric compound or components thereof into a disc annulus, around the exterior of the disc, and into the facet joints. As shown in FIG. 3, the method 300 comprises the steps of performing (310) a surgical procedure on a section of a spine, and reinforcing (320) a disc or facet joint relatively near the surgically treated area with an effective amount of a biocompatible matrix or biocompatible polymeric compound.

As used hereinafter, the term “biocompatible matrix” refers to materials that are cytocompatible, of interconnected open porosity, minimally- or non-inflammatory, nontoxic and generate minimal if any immune reaction when incorporated into a living being such as a human. Biological incorporation of a matrix depends, in part, upon the ability of cells to migrate into the matrix from the surrounding tissues and produce repair and or regeneration of the tissue defect. Thus, the structural and biochemical characteristics of the matrix may be further optimized to promote specific chemical, nutritional or tissue migration. Manipulation of these matrices are well known to those familiar with the art.

As used hereinafter, the term “biocompatible polymeric compound” refers to polymeric compounds that are, cytocompatible, biologically inert, degradable or nondegradable, non-inflammatory, nontoxic and generate minimal if any immune reaction when incorporated into a living being such as a human. A polymeric material is considered “biocompatible” if there is minimal fibrotic encapsulation on its surface subsequent to implantation, minimal inflammatory reaction, and no evidence of anaphylaxis during use. Thus, the material should elicit neither cell death through apoptosis or necrosis, an adverse humoral or cellular immune response, nor a nonspecific foreign body response. It should be noted, however, that materials meeting all the above-described criteria are relatively rare. Therefore, biocompatibility is more a matter of degree rather than an absolute state.

The biocompatible matrix or biocompatible polymeric compound may be injected as monomers that form the matrix or polymeric compound by polymerization and/or crosslinking at the injection site (in situ curable).

In essence, the injected, in situ curable, biocompatible matrix or biocompatible polymeric compound would (at least temporarily) provide limited re-hydration to the disc, while also providing a biologic milieu that may help to restore the health of the disc. The biocompatible matrix or biocompatible polymeric compound would also seal any previously undetected radial tears or pending weak zones in the annulus. By prophylactically treating discs and/or facet joints relatively near the surgically altered spinal section, or treating discs and facet joints that have been augmented with devices intended to prevent compressive overloading (in addition to the surgically altered spinal section), the procedure would delay or prevent the development of permanent or irreversible degenerative changes in the treated disc and/or facet joint, thus offering the spinal surgery patient a higher probability of long-term success for arresting disc and facet joint degeneration and prevention of latent adjacent disc problems. The effect of the treatment, such as re-hydration of a dehydrated disc, may be monitored using T2-weighted magnetic resonance imaging (MRI) or discography. In the presence of any ferro-magnetic implants, a CT or x-ray image could be utilized to evaluate disc height or changes in bone anatomy.

As used herein, the phrase “relatively near the surgically treated area” refers to a disc or facet joint that is within the proximity of the surgically treated area and is likely to be affected by the surgical treatment. The discs or facet joints relatively near the surgically treated area include the disc or facet joint adjacent to the surgically treated area and, some times, other discs or facet joints that are not more than three discs or facet joints away from the surgically treated area.

In one embodiment, the disc or facet joint relatively near the surgically treated area is reinforced by injecting the biocompatible matrix or biocompatible polymeric compound into and around the disc or facet joint. The spinal section may be repaired using any known spinal surgical procedures. Commonly used spinal surgical procedures include, but are not limited to, conventional open discectomy, mini-open discectomy, percutaneous discectomy, laminectomy, spinal fusion, artificial disc replacements (ADR), vertebral body replacements (VBR), partial vertebral body replacements (PVBR) and combinations thereof.

Discectomy is well known to one skilled in the art. It is typically used to decompress a nerve root or the spinal cord, to stabilize an unstable or painful segment of spine in combination with fusion, or to reduce a deformity in the spine. Discectomy can be done under either local, spinal or general anesthesia. During a conventional discectomy (also called “open discectomy”), the patient lays face down on the operating table, generally in a kneeling position. A small incision is made in the skin over the disc in need of treatment, such as a herniated disc, and the muscles over the spine are pulled back from the bone. A small amount of bone may be removed so the surgeon can see the compressed nerve. The herniated disc and any loose pieces are removed until they are no longer pressing on the nerve. Any bone spurs (osteophytes) are also taken out to make sure that the nerve is free of pressure.

Mini-open discectomy (also called micro-discectomy) involves no more than a 2-inch incision between the bones of the level involved and the operation involves removal of the soft tissues between the bones only, rather than any bony removal. Mini-open discectomy utilizes new techniques, such as the MRI scan, fluoroscopy x-rays in theatre, and better anesthetic techniques, which allow the operation to be undertaken through a much smaller incision, sometimes with the help of a microscope.

Discectomy may also be performed percutaneously using specially designed instruments. Percutaneous means “through the skin” or using a very small incision. Percutaneous discectomy is different from conventional open discectomy or micro-discectomy. There are several percutaneous procedures, all of which involve inserting small instruments between the vertebrae in order to gain access to the disc from the posterior side of the patient. X-ray monitoring is used during surgery to guide the movement of the surgical instruments. The surgeon can remove disc tissue by cutting it out, scraping in out, suctioning it out of the disc, or by using lasers to burn or evaporate the disc material.

Laminectomy is an adjunct to open discectomy, performed to permit the removal or reshaping of the lamina as part of a lumbar discectomy. During a laminectomy, the lamina of the vertebra is removed or trimmed to widen the spinal canal and create more space for the spinal nerves. It is a treatment for herniated disc, bulging or degenerated disc, such as spinal stenosis by relieving pressure on the spinal cord.

Spinal fusion is a “welding” process by which two or more of the vertebrae are fused together with bone grafts and utilizing internal devices such as screws and metal rods to stabilize bone structures until they can fuse. The surgery eliminates motion between vertebral segments, which may be desirable when abnormal motion is the cause of significant pain. It also stops the progress of a spinal deformity such as scoliosis. Spinal fusion is often used to treat injuries to the vertebral bodies, painful protrusions, degeneration and defects associated with more severely degenerated intervertebral disc, abnormal curvatures (such as scoliosis or kyphosis), and weak or unstable spine caused by infections or tumors. Spinal fusion, however, often causes loss of spinal mobility and flexibility, permanently altered motion characteristics and increases stress on and accelerates degeneration of adjacent discs that can lead to more pain and the need for more surgery.

Artificial disc replacement (ADR) offers a viable alternative to fusion that possibly avoids the shortcomings of fusion that can lead to more pain and the need for more surgery. An artificial disc (also called a disc replacement, disc prosthesis or spine arthroplasty device) is a device that is implanted into the spine to imitate the functions of a normal disc. ADR can be classified into two general types: total disc replacement and disc nucleus replacement. With a total disc replacement, all or most of the disc tissue is removed and a replacement device is implanted into the space between the vertebrae. The replacement devices (i.e. artificial discs) usually are made of metal or plastic-like (biopolymer) materials, or a combination of the two. By inserting an artificial disc instead of performing spinal fusion, there is the possibility of reducing damage to nearby discs and joints. This is because artificial disc replacement theoretically allows for motion preservation, near normal distribution of stress along the spine, and restoration of pre-degenerative disc height. However, this surgery is highly invasive and difficult to revise if the device should fail to perform as intended.

A promising alternative to fusion and ADR is the replacement of the nucleus pulposus alone. A synthetic nucleus replacement is implanted to recreate the biomechanical function of the intervertebral disc. With a disc nucleus replacement, only the center of the disc (the nucleus) is removed and replaced with an implant. The outer part of the disc (the annulus) is not removed, but requires that the annulus be surgically violated in order to place most if not all synthetic nucleus replacements.

Vertebral body replacements (VBR) are used in the thoracic and lumbar spine to replace a collapsed, damaged, or unstable vertebral body due to tumor or trauma (i.e. fracture). A VBR typically consists of a solid support (often called a cage or spacer) filled with bone cement. The solid support is generally made of artificial materials, such as titanium, ceramic, ceramic/glass, and carbon fiber. The solid support may be made expandable to permits an optimal, tight fit and correction of the deformity by in vivo expansion of the device.

In one embodiment, the biocompatible matrix or biocompatible polymeric compound is injected inside, or both inside and outside the disc and or facet joint, and relatively near the surgically treated spinal section, to reinforce the annulus wall and facet and may prevent the disc and facet joint from further degeneration by reinforcing the annulus by occluding and sealing disruptions, (at least temporarily) rehydrating the disc, and providing a biologic milieu and matrix for cell migration that may promote or accelerate healing. The biocompatible matrix or biocompatible polymeric compound can be injected immediately before or after the surgical procedure. The injection time is determined by the attending physician based on the nature and extent of the surgical procedure, the in vivo mixing and curing/setting times, the condition of the disc, and other patient concerns.

The biocompatible matrix or biocompatible polymeric compound can be non-degradable or degradable. A “degradable polymeric compound” is a polymeric compound that can be degraded and absorbed in situ in a living being such as human.

In one embodiment, the biocompatible polymeric compound is non-degradable. Non-degradable biocompatible polymeric compounds are typically synthetic polymers, such as degradable and nondegradable polyethylene glycol (PEG), poly(ethylene oxide) (PEO), poly(vinyl alcohol) (PVA), poly(vinylpyrrolidone) (PVP), poly(thyloxazoline) (PEOX), polyoxyethylene, polymethylene glycol, polytrimethylene glycols, polyvinyl-pyrrolidones, and derivatives thereof, polyoxyethylenepolyoxypropylene block polymers and copolymers, polyoxyethylene-polyoxypropylene block polymers and copolymers. The non-degradable polymers can be linear, branched, or crosslinked. In another embodiment, the biocompatible polymeric compound is degradable. The degradable and biocompatible polymeric compound minimizes potential problems, such as heat generation and undesirable biologic reactions from un-reacted monomers, that are typically associated with in situ polymerization of some non-degradable organic self-curing elastomers

Examples of the degradable biocompatible matrices include, but are not limited to, fibrin, type I collagen, type II collagen, type III collagen, fibronectin, laminin, hyaluronic acid (HA), hydrogel, pegylated hydrogel, chitosan, aliphatic polyesters, polylactides (PLA), polycaprolactone (PCL), polyglycolic acid (PGA), certain PEGs and Polyanhydrides, and combinations thereof.

In a preferred embodiment, the degradable biocompatible matrix is a fibrin sealant. The fibrin sealant is formed from fibrinogen and an activating agent that converts fibrinogen to fibrin. Fibrinogen can be autologous (i.e., from the patient to be treated), heterologous (i.e., from other human, pooled human supply, or non-human sources such as bovine and fish), or recombinant. Fibrinogen can be fresh or frozen. Fibrinogen is commercially available in freeze-dried form. Freeze-dried fibrinogen is typically reconstituted in a solution containing aprotinin (a polyvalent protease inhibitor which prevents premature degradation of the formed fibrin). In one embodiment, the reconstitution solution contains aprotinin at a concentration of 3000 KIU/ml. Typical concentrations for aprotinin range between 2000-4000 KIU/ml. Aprotinin may be derived from bovine lung, recombinant or synthetically derived.

The activating agent can be any agent that causes fibrinogen to form fibrin. Examples of the activating agent include, but are not limited to, thrombin and thrombin-like enzymes. Thrombin is an enzyme that converts fibrinogen to fibrin. Thrombin can be autologous (i.e., from the patient to be treated), heterologous (i.e., from other human, pooled human supply, or non-human source such as bovine, fish, various arachnids and other venomous species), or recombinant. Thrombin can be fresh or frozen. Thrombin is commercially available in freeze-dried form. Freeze-dried thrombin can be reconstituted in water or water containing calcium ions. In one embodiment, the reconstitution solution contains calcium chloride in the range of about 1 to 100 mmol/ml.

A thrombin-like enzyme is any enzyme that can catalyze the formation of fibrin from fibrinogen. A common source of thrombin enzymes is from bovines. Another common source of thrombin-like enzymes is snake venom (viperidae). Other sources of thrombin-like enzymes include various venomous marine life, such as jellyfish, sea snakes, cone shells, and sea urchins. Preferably, the thrombin-like enzyme is purified from the venom. Depending on the choice of thrombin-like enzyme, such thrombin-like enzyme can release fibrinopeptide A—which forms fibrin I—fibrinopeptide B—which forms des BB fibrin—or both fibrinopeptide A and B—which forms fibrin II. Thrombin-like enzymes that release fibrinopeptide A and B may do so at different rates. Thus, the resultant composition could be, for example, a mixture of fibrin II and fibrin I or a mixture of fibrin II and des BB fibrin.

TABLE 1 is a non-limiting list of the sources of the snake venoms that can be used with the herein disclosed methods, the name of the thrombin-like enzyme, and which fibrinopeptide(s) is released by treatment with the enzyme.

TABLE 1
Commonly used snake venoms

| | | Fibrinopeptide |
|---|---|---|
| Source | Name | Released |
| Agkistrodon acutus | Acutin | A |
| A. contortrix contortrix | Venzyme | B, (A)* |
| A. halys pallas | | B, (A)* |
| A. (Calloselasma) | Ancrod, Arvin | A |
| rhodostoma | | |
| Bothrops asper | Asperase | A |
| B. atrox, B. moojeni, | Batroxobin | A |
| B. maranhao | | |
| B. insularis | Reptilase | A, B |
| B. jararaca | Botropase/bothrombin | A |
| Lachesis muta muta | Defibrase | A, B |
| Crotalus adamanteus | Crotalase | A |
| C. durissus terrificus | | A |
| Trimeresurus flavoviridis | Flavoxobin/habutobin | A |
| T. gramineus | Grambin | A |
| Bitis gabonica | Gabonase | A, B |

*( ) means low activity.

For a review of thrombin-like enzymes from snake venoms, see H. Pirkle and K. Stocker, Thrombosis and Haemostasis, 65(4):444-450 (1991). The preferred thrombin-like enzymes are Batroxobin, especially from B. moojeni, B. maranhao and B. atrox; and Ancrod, especially from A. rhodostoma.

In the herein disclosed methods, fibrin formation begins immediately on contact of the fibrinogen and the activating agent, such as in the Y-connector of a dual syringe injection device such as that described by Miller et al. in U.S. Pat. No. 4,874,368, which is hereby incorporated by reference in its entirety. Another such dual syringe injection device is described in U.S. Provisional Patent Application Ser. No. 60/854,413, which is hereby incorporated by reference in its entirety.

The term “injecting” fibrin sealant as used herein thus encompasses any injection of components that form fibrin in the disc, facet joint(s), or surrounding spinal structures, including circumstances where a portion of the components react to form fibrin due to mixing prior to contact with or actual introduction into the disc. The herein disclosed methods include the sequential injection of the components of the fibrin sealant into the disc, facet joint(s), or surrounding spinal structures, such as by injecting the activating agent followed by the fibrinogen, or by injecting the fibrinogen followed by the activating agent. Likewise, the fibrinogen and the activating agent each can be intermittently injected into the disc, facet joint(s), or surrounding spinal structures.

Fibrin sealants mimic the final stage of the natural clotting mechanism. Fibrin sealants also provide a natural biologic matrix that promotes the healing process. Typically, such sealants entail the mixing of a fibrinogen component with an activating enzyme such as thrombin. To increase biocompatibility of the sealant with host tissue, various components may be supplied endogenously from host body fluids. Combining the reconstituted components produces a viscous solution that quickly sets into an elastic coagulum. A method of preparing a conventional fibrin sealant is described by J. Rousou, et al. (J. Rousou, et al. Journal of Thoracic and Cardiovascular Surgery, 1989, 97:194-203). Cryoprecipitate derived from source plasma is washed, dissolved in a buffer solution, filtered and freeze-dried. The freeze-dried fibrinogen is reconstituted in solution containing a fibrinolysis inhibitor. The solution is stirred and heated to a temperature of about 37° C. Each solution (the thrombin and fibrinogen solutions) is drawn up in a syringe and mounted on a Y-connector to which a needle is attached for delivery of the combined solution (see, e.g. the Duploject™ device, from ImmunoAG, Vienna, Austria). Thus, mixing of the components only occurs during the delivery process, which facilitates clot formation only at the desired site of application. The components should be injected sufficiently quickly to avoid the passage becoming blocked due to fibrin formation in the needle and/or Y-connector.

In one embodiment, a dual-syringe injector is used and the mixing of the fibrin sealant components at least partially occurs in the Y-connector and in the needle mounted on the Y-connector, with the balance of the clotting occurring in the disc, facet joint(s), or surrounding spinal structures. This method of preparation facilitates the formation of a fibrin clot at the desired site in the disc, facet joint(s), or surrounding spinal structures during delivery, or immediately thereafter.

In another embodiment, a multi-syringe injector is used. The injector has two syringes for mixing fibrin sealant components during injection in a Y-connector or a coaxial needle, and additional syringe(s) for introducing additional additives into the Y-connector or a coaxial needle during injection.

In another embodiment, freeze-dried fibrinogen is reconstituted to a concentration of about 75-115 mg/ml, and freeze-dried thrombin is reconstituted separately to a final concentration of about 400-600 I.U./ml, preferably in a concentration of about 1-10 I.U./ml and more preferably at about 4-5 I.U./ml. Freeze-dried fibrinogen and freeze-dried thrombin are available in kit form from manufacturers such as Baxter under names such as TISSEEL™. These two fibrin sealant components can be prepared in about 2 ml samples each to yield approximately 4 ml of total sealant (reconstituted fibrinogen plus reconstituted thrombin). In another embodiment, at least one of the reconstituted fibrinogen and thrombin is reconstituted using a solution containing at least one additive. A preservative-free reconstituting solution may be used, but is not required.

The point, or points, of injection (e.g., at the tip of a spinal needle) can be in the nucleus pulposus, within the annulus fibrosus, or outside the annulus fibrosus. If the injection occurs in the nucleus pulposus, the injected components may form a patch at the interface between the nucleus pulposus and the annulus fibrosus, or, more commonly, the components flow into the defect(s) (e.g., fissures) of the annulus fibrosus and potentially “overflow” into the interdiscal space. Over-pressurizing the disc when injecting the components into the disc should be avoided.

The point, or points, of injection (e.g., at the tip of a spinal needle) can be in the facet joint(s), or surrounding spinal structures. If the injection occurs in the facet joints, the injected components may form a patch at the interface between the facets, and/or within the fibrous tissues of the synovial joint between the superior articular process of one (lower) vertebra and the inferior articular process of the adjacent (higher) vertebra. There are two facet joints in each vertebral motion segment.

Given the natural function of fibrin sealant within the body as a natural biologic matrix that promotes the healing process, it is also envisioned that the point, or points, of injection (e.g., at the tip of a spinal needle) can be at any number of points in or around the spine, such as the insertion point(s) of muscles, tendons and/or ligaments. During the course of surgery around the spine, particularly mini-open and percutaneous surgery, many surrounding tissues are damaged from stretching and tearing, and would benefit from the natural biologic matrix provided by fibrin sealant, should it be applied via injection during the repair phase of the surgery. This same concept could naturally be extended to many other mini-open and percutaneous surgeries.

The fibrin sealant may be administered with an anesthetic, such as a local anesthetic. Representative local anesthetics include but are not limited to lidocaine HCL (often sold in concentrations of 1.5 percent or 4 percent), SARAPIN anesthetic (a sterile aqueous solution of soluble salts and bases from Sarraceniaceae (Pitcher Plant), and bupivacaine HCL (also known as marcaine, which is often sold in concentrations of 0.5 percent and 0.75 percent). The chemical name for lidocaine is alpha-diethylaminoaceto-2,6-xylidide, and the IUPAC name is 2-(diethylamino)-N-(2,6-dimethylphenyl)acetamide. The chemical name for bupivicaine is 1-butyl-N-(2,6-dimethylphenyl)-2-piperidinecarboxamide, sometimes referred to as 1-butyl-2′,6′-pipecoloxylidide monohydrochloride, having registry number 14252-80-3. Alternatively, procaine (2-diethylaminoethyl 4-aminobenzoate hydrochloride) or other local anesthetic can be employed. Among the local anesthetics, bupivacaine is preferred. Combinations of anesthetics also can be used. The anesthetic can be injected with the fibrin sealant. Alternatively, the anesthetic can be injected separately, either before or after the injection of the fibrin sealant. Preferably, the anesthetic is injected prior to, or simultaneously with, the injection of the fibrin sealant.

In one embodiment, a solution containing a local anesthetic is used to reconstitute the fibrinogen or the activating agent. In another embodiment, the fibrinogen or the activating agent is reconstituted without an anesthetic, and the anesthetic is then added to the reconstituted fibrinogen or the activating agent.

In general, the amount of anesthetic used should be chosen so as to be effective in alleviating the pain of injection when the sealant is injected or otherwise introduced into the disc, facet joint(s), or surrounding spinal structures. In one embodiment, a solution containing about 0.1 to about 10 percent by weight of anesthetic is used. The injected volume of the anesthetic solution can vary widely, such as from about 0.1 ml to about 5 ml, depending one the mode of injection.

The fibrin sealant may be administered with one or more additives. As used herein, the term “additives” includes antibiotics; antiproliferative, cytotoxic, and antitumor drugs including chemotherapeutic drugs; analgesic; antiangiogen; antibody; antivirals; cytokines; colony stimulating factors; proteins; chemoattractants; chelating agents such as EDTA; histamine; antihistamine; erythropoietin; antifungals; antiparasitic agents; non-corticosteroid anti-inflammatory agents; anticoagulants; anesthetics including local anesthetics such as lidocaine and bupivicaine; analgesics; oncology agents; cardiovascular drugs; vitamins and other nutritional supplements; hormones; glycoproteins; fibronectin; peptides including polypeptides and proteins; interferons; cartilage inducing factors; protease inhibitors; vasoconstrictors, vasodilators, demineralized bone or bone morphogenetic proteins; hormones; lipids; carbohydrates; proteoglycans such as aggrecan (chondrotin sulfate and deratin sulfate), versican, decorin, and biglycan; antiangiogenins; antigens; deminerized bone matrix (DBM); hyaluronic acid and salts and derivatives thereof; polysaccharides; cellulose compounds such as methyl cellulose, carboxymethyl cellulose, and hydroxy-propylmethyl cellulose and derivatives thereof; antibodies; gene therapy reagents; genetically altered cells, stem cells including mesenchymal stem cells with transforming growth factor, and/or other cells; cell growth factors to promote rehabilitation of damaged tissue and/or growth of new, healthy tissue such as BMP7 and BMP2; type I and II collagen; elastin; sulfated glycosaminoglycan (sGAG), glucosamine sulfate; pH modifiers; methylsulfonylmethane (MSM); osteogenic compounds; osteoconductive compounds; plasminogen; nucleotides; oligonucleotides; polynucleotides; polymers; osteogenic protein 1 (OP-1 including recombinant OP-1); LMP-1 (Lim Mineralization Protein-1); cartilage including autologous cartilage; oxygen-containing components; enzymes such as, for example, peroxidase, which mediate the release of oxygen from such components; melatonin; vitamins; and nutrients such as, for example, glucose or other sugars. In one embodiment, the additive is a growth factor that promotes rehabilitation of the damaged tissues.

Any of the aforementioned additives may be added to the fibrin sealant separately or in combination. For example, one or more of these additives can be injected with the fibrin sealant. Combinations of these additives can be employed and different additives can be used in the solutions that are used to reconstitute the fibrinogen or the activating agent. In one embodiment, a solution containing a local anesthetic and glucosamine sulfate is used to reconstitute the fibrinogen, and a solution containing type II collagen is used to reconstitute the activating agent. Likewise, one or more of these additives can be injected separately, either before or after the injection o the fibrin sealant.

For solutions containing an incompletely water-soluble additive(s), an anti-caking agent such as polysorbate, may be added to facilitate suspension of this component.

The fibrin sealant will generally be used in an amount effective to achieve the intended result, i.e., delay or prevent degeneration of the disc and/or facet joint(s), or surrounding spinal structures relatively near the surgically treated spinal section. The effective amount of the fibrin sealant administered will depend upon a variety of factors, including, for example, the type of surgical procedure used for treating the spinal section, the site of the surgical procedure, the mode of administration, whether the desired benefit is prophylactic or therapeutic, the condition of the adjacent disc, facet joint(s), or surrounding spinal structures, the age and weight of the patient, and the bioavailability of the particular active agent. Determination of an effective dosage is well within the capabilities of those skilled in the art.

Effective dosages may be estimated initially from in vitro assays and in vivo animal models. Suitable animal models of degenerative disc diseases and discogenic pain include rat and rabbit models described in, for example, Norcross et al. An in vivo model of degenerative disc disease, J. Orthopaedic Research, 2003, 21:183-188; and Larson et al., Biologic Modification of Animal Models of Intervertebral Disc Degeneration, The Journal of Bone and Joint Surgery (American), 2006, 88:83-87. Ordinarily, skilled artisans can routinely adapt such information to determine dosages suitable for human administration.

The fibrinogen is typically used in a concentration range of 50-150 mg/ml. The amount of activating agent such as thrombin can be varied to reduce or lengthen the time to complete fibrin formation. The fibrinogen is typically in the range 50 to 150 mg/ml and the thrombin is typically in the range 4 IU/ml to 600 IU/ml. In general, the higher level of thrombin per unit amount of fibrinogen, the faster fibrin formation occurs. If slower fibrin formation is desired, then less thrombin is used per unit fibrinogen. The fibrin formation time (i.e., the polymerization time of the fibrinogen) may be important for controlling the time at which the clot forms so as to ensure the fibrin sealant sets up at the proper site and time in the body rather than setting-up prematurely. Additionally, the aggressiveness of the mixing of the components plays a significant role in the setting time. The method of delivery can have a significant effect on clot time, uniformity of mixing density and strength of clot. Likewise, varying the fibrinogen concentration may change the density of the combined components, which may be important for controlling flow through a long conduit such as a catheter into the body. The use of calcium ions (such as from calcium chloride) in the thrombin component solution will affect the strength of the fibrin so formed, with increasing amounts of calcium ions increasing the strength of the fibrin clot.

For intradiscal injections, the total volume of the injection is limited. Typically, a total volume of 1-5 ml fibrin sealant is used for intradiscal and facet joint injections. A larger volume of the fibrin sealant may be applied extradiscally and for surrounding tissues. Injection of fibrin sealant into (within) blood vessels is to be avoided.

The dosage and volume of fibrin sealant may be adjusted individually to provide local concentrations of the agents that are sufficient to maintain a protective or therapeutic effect. For example, the fibrin sealant may be administered in a single injection or by sequential injections. The injection may be repeated periodically. Skilled artisans will be able to optimize effective local dosages and the injection regimen without undue experimentation.

Preferably, the fibrin sealant will provide a protective or therapeutic benefit without causing substantial toxicity. Toxicity of the fibrin sealant may be determined using standard pharmaceutical procedures. The dose ratio between toxic and protective/therapeutic effect is the therapeutic index. Agents that exhibit high protective/therapeutic indices are preferred.

Preferably, a non-iodinated contrast agent may be used in conjunction with the injection of the fibrin sealant to ensure the correct placement at the site and avoidance of blood vessels. The contrast agent may be injected prior to injection of the fibrin sealant. Alternatively, the contrast agent may be included in the fibrinogen component or the activating agent component that is injected into the disc. Contrast agents and their use are well known to those skilled in the art.

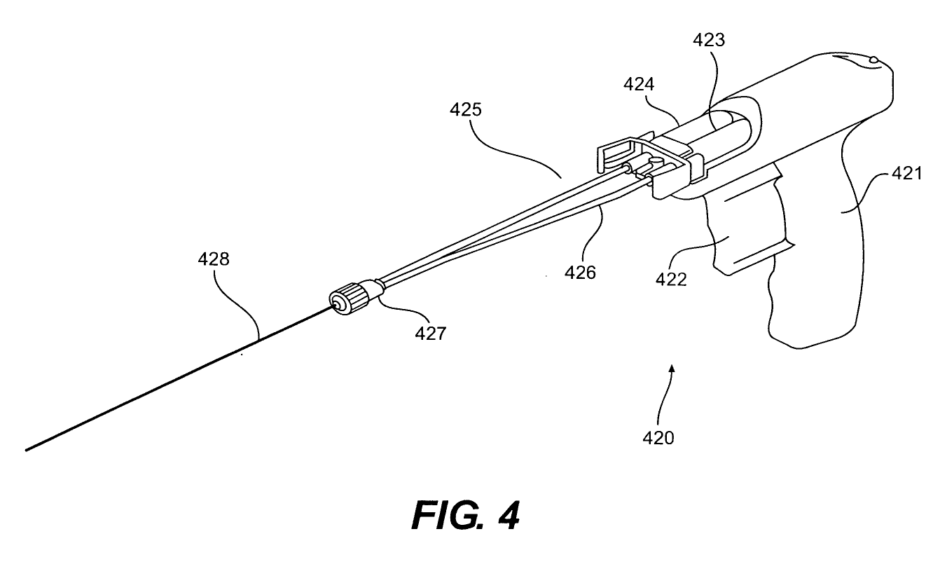

The fibrin sealant may be injected into or outside the disc using a delivery device such as that shown in FIG. 4. Delivery device 420 includes main housing 421 into which are inserted fibrinogen capsule 423 and thrombin capsule 424. Trigger 422, in conjunction with a pressure monitor (not shown) controls injection of the fluids. Attached to the capsules 423, 424 is an inner needle assembly including delivery tubes 425 and 426, (connected to an inner, coaxial needle, (not shown), within the outer needle 428). Connector 427 serves to connect the delivery tubes 425, 426 and the inner coaxial needle to the outer needle 428.

One skilled in the art would understand that the above-described injection procedures and delivery devices also apply to injection of other degradable biocompatible polymeric compounds,and non-degradable biocompatible polymeric compounds.

In another embodiment, the reinforcing step is performed in conjunction with the installation of a dynamic stabilization or “flexible” spine stabilization systems (which would include interspinous and facet stabilization systems). The dynamic stabilization or “flexible” spine stabilization systems provide additional medial/lateral (M/L) and anterior/posterior (A/P) stability to the spine and adjacent segments. Examples of the dynamic stabilization or “flexible” spine stabilization systems include, but are not limited to, the Dynesis® System (Zimmer, Inc., Warsaw, Ill.), the Wallis® System (Abbott Laboratories, Abbott Park, Ill.), The X-STOP™ System (St. Francis Medical Technologies, Inc., Alameda, Calif.), the DIAM™ system (Medtronic Sofamor Danek, Minneapolis, Minn.), and Colfex™ (Paradigm Spine, LLC. New York, N.Y.).

In another embodiment, the prophylactic treatment is applied to the facet joints of the surgically treated disc and adjacent discs at the same time. It is well documented that facet joints share a similar fate to spinal discs in cases of adjacent disc disease. Assuming that the facet joint degrades from over-pressurization and wear due to an imbalance in the spinal architecture resulting from various forms of degenerated disc diseases and/or various arthritic conditions, one would expect to see bone-on-bone, tears in the joint capsule and innervations of the capsule. It is anticipated that application of a biocompatible polymeric compound to these joints would have a similar sealing and healing effect to the facet joint, as well as other surrounding tissues.

Also disclosed is a kit for prophylactically treating discs, facet joint(s), or surrounding spinal structures relatively near a spinal section that requires surgery. In one embodiment, the kit comprises (1) components needed for the formation of a biocompatible matrix or biocompatible polymeric compound, (2) needles and/or catheters for delivering the biocompatible matrix or biocompatible polymeric compound, or components thereof, into a disc annulus and around the exterior of the disc, facet joint(s), or surrounding spinal structures, (3) a delivery device for injecting the biocompatible matrix or biocompatible polymeric compound or components thereof, and (4) a spine stabilization device.

Examples of the spine stabilization devices include, but are not limited to, the Dynesis® system, the Wallis® system, the X-STOP™ system, the DIAM™, the Intraspinous U™, the CoFleX™, BioDisc™, DASCOR™, Prosthetic Disc Nucleus (PDN), NeuDisc™, NuCore™, Aguarelle™, Newcleus™, vertebral body replacements (VBR), and partial VBR (PVBR).

In one embodiment, the kit further comprises a fifth component selected from the group consisting of antibiotics; antiproliferative, cytotoxic, and antitumor drugs including chemotherapeutic drugs; analgesic; antiangiogen; antibody; antivirals; cytokines; colony stimulating factors; proteins; chemoattractants; Chelating agents such as EDTA, histamine; antihistamine; erythropoietin; antifungals; antiparasitic agents; non-corticosteroid anti-inflammatory agents; anticoagulants; anesthetics; analgesics; oncology agents; cardiovascular drugs; vitamins and other nutritional supplements; hormones; glycoproteins; fibronectin; peptides including polypeptides and proteins; interferons; cartilage inducing factors; protease inhibitors; vasoconstrictors, vasodilators, demineralized bone or bone morphogenetic proteins; hormones; lipids; carbohydrates; proteoglycans; antiangiogenins; antigens; demineralised bone matrix (DBM); hyaluronic acid and salts and derivatives thereof; polysaccharides; cellulose compounds and derivatives thereof; antibodies; gene therapy reagents; genetically altered cells, stem cells including mesenchymal stem cells with transforming growth factor, and/or other cells; cell growth factors; type II collagen; elastin; sulfated glycosaminoglycan (sGAG), glucosamine sulfate; pH modifiers; methylsulfonylmethane (MSM); osteogenic compounds; osteoconductive compounds; plasminogen; nucleotides; oligonucleotides; polynucleotides; polymers; osteogenic protein 1 (OP-1 including recombinant OP-1); LMP-1 (Lim Mineralization Protein-1); cartilage; oxygen-containing components; enzymes; melatonin; vitamins; and nutrients.

The disclosed method may be better understood by reference to the following examples, which are representative and should not be construed to limit the scope of the claims hereof.

### EXAMPLES

### Example 1

### Injection of Fibrin Sealant with a Dual-Syringe Injector

Injection of the fibrin sealant involves several steps, which are outlined below. The example presented is based on use of the delivery device 420 shown in FIG. 4.

### Pre-Medication

As a first step, intravenous antibiotics are administered 15 to 60 minutes prior to commencing the procedure as prophylaxis against discitis. Patients with a known allergy to contrast medium should be pre-treated with H1 and H2 blockers and corticosteroids prior to the procedure in accordance with International Spine Intervention Society (ISIS) recommendations. Sedative agents may be administered but the patient should remain awake during the procedure and capable of responding to pain from pressurization of the disc. If the fibrin sealant is injected immediately after a surgical procedure (e.g., discectomy), the pre-medication step may not be necessary.

### Preparation

The injection procedure should be performed in a suite suitable for aseptic procedures and equipped with fluoroscopy (C-arm or two-plane image intensifier) and an x-ray compatible table to allow visualization of needle placement.

Local anesthetic for infiltration of skin and deep tissue and nonionic contrast medium with 10 mg per cc of antibiotic should be available for this procedure.

### Preparation of the Fibrin Sealant

Preparation of the fibrin sealant may require approximately 25 minutes. In an embodiment, freeze-dried fibrinogen and thrombin are reconstituted in a fibrinolysis inhibitor solution and a calcium chloride solution, respectively. The reconstituted fibrinogen and thrombin solutions are then combined upon delivery with the delivery device 420 to form the fibrin sealant within the treated disc.

### Preparation of the Delivery Device

Maintaining a sterile environment, the delivery device 420 is assembled and checked for function in preparation for the reconstituted thrombin and fibrinogen component solutions to be transferred into the device.

### Patient Positioning and Skin Preparation

The patient should lie on a radiography table in either a prone or oblique position depending on the physician's preference. By means of example for a lumbar disc treatment, the skin of the lumbar and upper gluteal region should be prepared as for an aseptic procedure using non-iodine containing preparations.

### Target Identification

For intradiscal injections, disc visualization and annulus fibrosus puncture should be conducted according the procedures used for provocation discography. The targeted disc should be approached from the side opposite of the patient's predominant pain. If the patient's pain is central or bilateral, the target disc can be approached from either side.

An anterior-posterior (AP) image of the lumbar spine is obtained such that the x-ray beam is parallel to the inferior vertebral endplate of the targeted disc. The beam should then be angled until the lateral aspect of the superior articular process of the target segment lies opposite the axial midline of the target disc. The path of the intradiscal needle should be parallel to the x-ray beam, within the transverse mid-plane of the disc, and just lateral to the lateral margin of the superior articular process.

### Placement of the Intradiscal Needle

The intradiscal needle is specifically designed to facilitate annular puncture and intradiscal access for delivery of the fibrin sealant. The intradiscal needle is manufactured with a slight bend in the distal end to enhance directional control of the needle as it is inserted through the back muscles and into the disc.

The intended path of the intradiscal needle is anesthetized from the subcutaneous tissue down to the superior articular process. The intradiscal needle initially may be inserted under fluoroscopic visualization down to the depth of the superior articular process. The intradiscal needle will be then slowly advanced through the intervertebral foramen while taking care not to impale the ventral ramus. If the patient complains of radicular pain or paraesthesia, advancement of the needle must be stopped immediately and the needle must be withdrawn approximately 1 cm. The path of the needle should be redirected and the needle slowly advanced toward the target disc. Contact with the annulus fibrosus will be noted as a firm resistance to continued insertion of the intradiscal needle. The needle will be then advanced through the annulus to the center of the disc. Placement of the needle is confirmed with both AP and lateral images. The needle tip should lie in the center of the disc in both views.

Once the needle position is confirmed, a small volume of non-ionic contrast medium may be injected into the disc. A minimal volume of contrast may be injected to insure avascular flow of the contrast media. If vascular flow is seen, the intradiscal needle should be repositioned and the contrast injection repeated.

### Loading the Delivery System

After correct placement of the intradiscal needle is confirmed, the reconstituted fibrinogen and thrombin solutions are transferred into the appropriate chambers of the delivery device 420.

### Attaching the Inner Needle Assembly and Intradiscal Needle

The inner needle assembly next is attached to the delivery device 120, and air is expelled from the device. The inner needle assembly with the inner coaxial needle, is next inserted into the intradiscal needle which is already in the center of the target disc, creating a coaxial delivery needle.

### Delivery of the Fibrin Sealant

Placement of the intradiscal needle tip in the center of the target disc is reconfirmed with AP and lateral images. The trigger is then depressed to begin application of fibrin sealant to the disc. Pressure should be monitored constantly when squeezing the trigger. To prevent over-pressurization of the disc, pressure should not exceed 100 psi (6.8 atm) for a lumbar disc.

Each full compression of the trigger will deliver approximately 1 mL of the fibrin sealant to the disc. When the trigger is released, it automatically resets to the fully uncompressed position. Once all of the fibrin sealant has been delivered, the trigger will stop advancing.

Periodic images of the disc should be taken during application of the fibrin sealant to insure that the intradiscal needle has not moved from the center of the disc.

Application of the fibrin sealant to the disc should continue until one of the three following events occurs.
1. The total desired volume of the fibrin sealant is delivered to the disc, usually between 1-3 ml, (accounting for any losses within the tubing, needle, system, etc).
2. Continued application of the fibrin sealant would require pressures above 100 psi (6.8 atm).
3. The patient cannot tolerate continuation of the procedure.

After the application of the fibrin sealant is stopped, the intradiscal needle is carefully removed from the patient. Patient observation and vital signs monitoring will be performed for about 20-30 minutes following the procedure.

Extradiscal injection of the fibrin sealant (i.e., injection of fibrin sealant to the exterior of the weakened portion of the herniated disc) may also be carried out using procedures described above. An additional 1-3 ml of fibrin sealant, or the remaining amount available in the delivery device, should be delivered to the external area of the disc that had received surgical decompression. If appropriate, additional amounts of fibrinogen and thrombin may be (prepared and) loaded into the delivery device and delivered to the extradiscal area of the disc annulus. Additionally, fibrin sealant may be injected into other tissues of surrounding spinal structures where benefit from the natural healing milieu may be obtained.

### Example 2

### Re-Hydration of Spinal Disc after Injection of Fibrin Sealant

A 66 year old male patient was diagnosed with degenerative disc disease and a herniated L4/L5 disc in December of 2004. At the time of the original diagnosis, discography also revealed IDD in discs L2/L3 and L3/L4, indicating leaking discs with a corresponding loss of disc height. In January, 2005, he received a partial discectomy to decompress the spinal cord and nerve roots on L4/L5 with the Stryker DeKompressor. He received immediate sealant injection treatment on date of surgery in the L4/L5 disc and around the exterior surgical site. He received 3 cc of fibrin sealant in the L/4/L5 disc nucleus and around the exterior surgical site.

In addition, the patient also received sealant injections into the L2/L3 and L3/L4 discs to treat the discogenic pain, (IDD). He received 1 cc each, injected into the nucleus of the L2/L3 and L3/L4 discs. (5 cc total for patient). A subsequent discography procedure has revealed a complete sealing of all of the treated discs, along with a return of normal disc height and a complete cessation of pain.

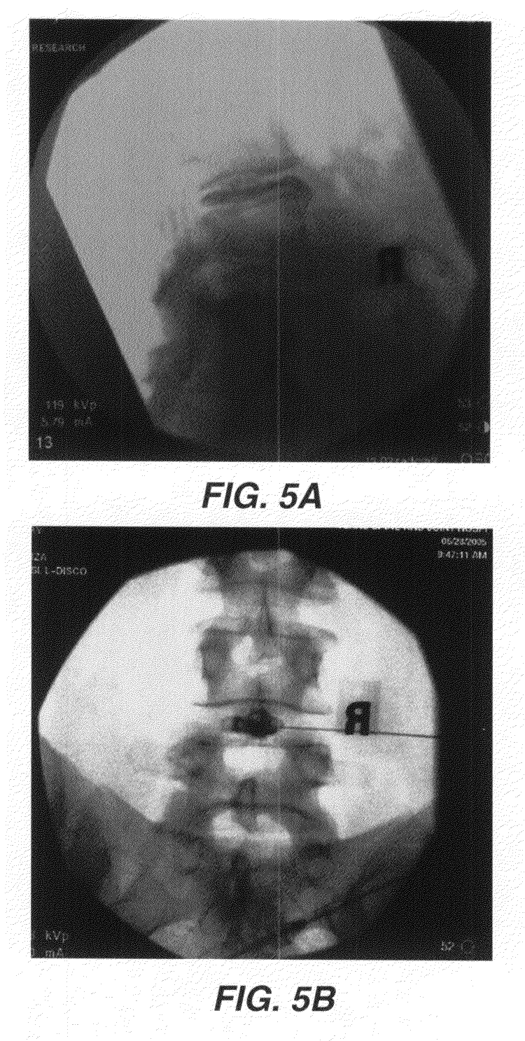

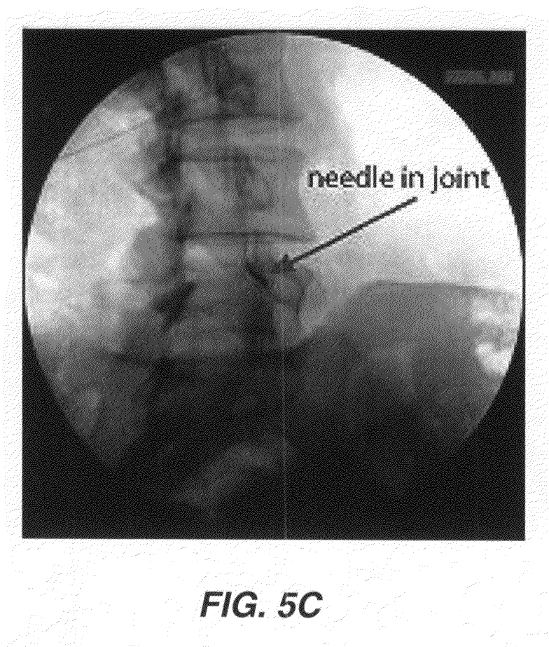

The intradiscal injection of fibrin sealant led to re-hydration of the treated disc. FIG. 5A shows a medial/lateral view of the disc prior to treatment with fibrin sealant, demonstrating annular tears and dehydration. FIG. 5B shows an anterior/posterior view of the same disc at 6 months after the fibrin sealant treatment, demonstrating re-hydration and improved annular structure. The positive results have been maintained for the 2+ years since his procedure, with no further treatment needed.

### Example 3

### Injection into Facet Joints

Injection of the facet joints is performed using the device and procedures described in Example 1. FIG. 5C is a fluoroscopic x-ray of a facet joint injection. Briefly, . . . Following the surgical treatment of the affected areas of the spine, (i.e.: discectomy, fusion, ADR, VBR or PVBR), the patient is placed in such a way that the physician can best visualize the facet joints using x-ray guidance. Next, the physician directs the needle, using x-ray guidance into the facet joint(s). A small amount of contrast (dye) is injected to insure proper needle position inside the joint space. Then, an effective amount of the biocompatible matrix or biocompatible polymeric compound is injected. One or several joints may be injected depending on location of the patients usual pain, the degree of surrounding joint degradation and the degree of involvement of the surgically treated spinal area near the facet joints being treated.

### Example 4

### Stabilization of Discs or Facet Joints Adjacent to a Surgically Treated Spinal Section with a Dynamic Stabilization or Flexible Spinal System and Injection of Fibrin Sealant

A patient requiring spinal surgery will be prepared for spinal surgery. Upon exposure of the spine, the intended procedure, (i.e.: discectomy, fusion, ADR, VBR or PVBR), would be performed, and possibly followed by the installation of the Dynamic Stabilization or Flexible Spinal System. Immediately prior to making final adjustments of the Dynamic Stabilization or Flexible Spinal System, discs, facet joints and damaged tissues that are immediately adjacent to or relatively near the specifically treated disc, would be injected with fibrin sealant using procedures described in Example 1. Following completion of the injections, any final adjustments would be made to the Dynamic Stabilization or Flexible Spinal System and the wound would be closed in the normal fashion.

The herein described methods may be used to address various conditions through use of the surgical procedure and fibrin sealant. The disclosure references particular means, materials and embodiments elaborating limited application of the claims. The claims are not limited to these particulars and applies to all equivalents. Although the claims make reference to particular means, materials and embodiments, it is to be understood that the claims are not limited to these disclosed particulars, but extend instead to all equivalents.

What is claimed is:

1. A method for prophylactically treating discs and facet joints, said method comprising:
performing a surgical procedure on a section of a spine; and
reinforcing a disc or a facet joint with an effective amount of a biocompatible matrix or biocompatible polymeric compound,
wherein said disc or said facet joint is relatively near said section of the spine.

2. The method of claim 1, wherein said reinforcing step comprises injecting said biocompatible polymeric compound into said disc or said facet joint.

3. The method of claim 2, wherein said biocompatible polymeric compound is a biocompatible degradable polymeric compound.

4. The method of claim 2, wherein said biocompatible polymeric compound is a biocompatible non-degradable polymeric compound.

5. The method of claim 2, wherein said reinforcing step comprises injecting said biocompatible polymeric compound into and outside said disc.

6. The method of claim 5, wherein said biocompatible polymeric compound is a biocompatible degradable polymeric compound.

7. The method of claim 5, wherein said biocompatible polymeric compound is a biocompatible non-degradable polymeric compound.

8. The method of claim 2, wherein said reinforcing step comprises injecting said biocompatible polymeric compound into and outside said facet joint.

9. The method of claim 8, wherein said biocompatible polymeric compound is a biocompatible degradable polymeric compound.

10. The method of claim 8, wherein said biocompatible polymeric compound is a biocompatible non-degradable polymeric compound.

11. The method of claim 1, further comprising the step of reinforcing said section of the spine with an effective amount of said biocompatible polymeric compound after the surgical procedure.

12. The method of claim 11, wherein said section of the spine comprises a degenerated disc.

13. The method of claim 12, further comprising the step of reinforcing the degenerated disc by injecting said biocompatible polymeric compound into and outside a surgically treated disc area.

14. The method of claim 13, wherein said biocompatible polymeric compound is a biocompatible degradable polymeric compound.

15. The method of claim 13, wherein said biocompatible polymeric compound is a biocompatible non-degradable polymeric compound.

16. The method of claim 11, wherein said section of the spine comprises a degenerated facet joint.

17. The method of claim 16, further comprising the step of reinforcing the degenerated facet joint by injecting said biocompatible polymeric compound into and outside a surgically treated facet joint area.

18. The method of claim 17, wherein said biocompatible polymeric compound is a biocompatible degradable polymeric compound.

19. The method of claim 17, wherein said biocompatible polymeric compound is a biocompatible non-degradable polymeric compound.

20. The method of claim 3, wherein said biocompatible degradable polymeric compound is a fibrin sealant.

21. The method of claim 20, wherein said fibrin sealant is produced by mixing fibrinogen with an activating agent during injection.

22. The method of claim 21, wherein said activating agent is thrombin or a snake venom derivative.

23. The method of claim 21, wherein said fibrinogen is autologous fibrinogen.

24. The method of claim 21, wherein said fibrinogen is heterologous fibrinogen.

25. The method of claim 20, wherein said fibrin sealant is injected with an anesthetic.

26. The method of claim 20, wherein said fibrin sealant is injected with at least one additive.

27. The method of claim 26, wherein said at least one additive is selected from the group consisting of antibiotics; antiproliferative, cytotoxic, antitumor drugs, chemotherapeutic drugs; analgesic; antiangiogen; antibody; antivirals; cytokines; colony stimulating factors; proteins; chemoattractants; chelating agents; histamine; antihistamine; erythropoietin; antifungals; antiparasitic agents; non-corticosteroid anti-inflammatory agents; anticoagulants; anesthetics; analgesics; oncology agents; cardiovascular drugs; vitamins and other nutritional supplements; hormones; glycoproteins; fibronectin; peptides including polypeptides and proteins; interferons; cartilage inducing factors; protease inhibitors; vasoconstrictors, vasodilators, demineralized bone or bone morphogenetic proteins; hormones; lipids; carbohydrates; proteoglycans; antiangiogenins; antigens; demineralised bone matrix (DBM); hyaluronic acid and salts and derivatives thereof; polysaccharides; cellulose compounds and derivatives thereof; antibodies; gene therapy reagents; genetically altered cells, stem cells, mesenchymal stem cells with transforming growth factor; cell growth factors; type II collagen; elastin; sulfated glycosaminoglycan (sGAG), glucosamine sulfate; pH modifiers; methylsulfonylmethane (MSM); osteogenic compounds; osteoconductive compounds; plasminogen; nucleotides; oligonucleotides; polynucleotides; polymers; osteogenic protein 1 (OP-1), recombinant OP-1); Lim Mineralization Protein-1(LMP-1); cartilage; oxygen-containing components; enzymes; melatonin; vitamins; and nutrients.

28. The method of claim 20, wherein said fibrin sealant is injected in the presence of aprotinin and calcium ions.

29. The method of claim 20, wherein said injection of said fibrin sealant is performed using a dual syringe injector.

30. The method of claim 20, wherein said injection of said fibrin sealant is performed using a multiple-syringe injector.

31. The method of claim 29, wherein said injecting step comprises:
inserting an introducer needle having a tip into a position adjacent to or inside the disc;
inserting a second needle or a polymeric catheter through the introducer needle up to but not beyond the tip of the introducer needle; and
simultaneously injecting a first fibrin sealant component through the introducer needle and a second fibrin sealant component through the second needle or polymeric catheter,
wherein said first fibrin sealant component is fibrinogen and said second fibrin sealant component is an activating agent, or
wherein said first fibrin sealant component is an activating agent and said second fibrin sealant component is fibrinogen.

32. The method of claim 31, wherein said injection is pressure-monitored.

33. The method of claim 1, wherein said disc is a lumbar disc.

34. The method of claim 1, wherein said facet joint is a lumbar facet joint.

35. The method of claim 1, wherein said disc is a cervical disc.

36. The method of claim 1, wherein said facet joint is a cervical facet joint.

37. The method of claim 1, wherein said disc is a thoracic disc.

38. The method of claim 1, wherein said facet joint is a thoracic facet joint.

39. The method of claim 2, further comprising injecting a contrast agent into said disc before the injection of said biocompatible polymeric compound.

40. The method of claim 1, wherein said section of the spine is treated by a surgical procedure selected from the group consisting of conventional open discectomy, mini-open discectomy, percutaneous discectomy, spinal fusion, artificial disc replacements (ADR), vertebral body replacements (VBR), partial vertebral body replacements (PVBR) and combinations thereof.

41. The method of claim 2, wherein said injection to said disc or said facet joint is performed immediately after said surgical procedure.

42. The method of claim 1, wherein said disc or said facet joint is clearly compromised or at high risk of degeneration.

43. A kit for prophylactically treating discs and facet joints relatively near a spinal section that requires surgery, said kit comprising:
components needed for the formation of a biocompatible matrix or a biocompatible polymeric compound;
a needle, a catheter, or both for delivering the biocompatible matrix or biocompatible polymeric compound, or components thereof, into a disc annulus, around the exterior of the disc, or into a facet joint;
a delivery device for injecting the biocompatible matrix and biocompatible polymeric compound or components thereof, and
a spine stabilization device.

44. The kit of claim 43, wherein said spine stabilization device is selected from the group consisting of dynamic stabilization systems, flexible spinal system, artificial disc replacement (ADR), vertebral body replacements (VBR), partial VBR (PVBR), bone graft materials for fusion, and synthetic spacer materials for fusion.

45. The kit of claim 43, further comprising a component selected from the group consisting of antibiotics; antiproliferative, cytotoxic, antitumor drugs, chemotherapeutic drugs; analgesic; antiangiogen; antibody; antivirals; cytokines; colony stimulating factors; proteins; chemoattractants; chelating agents; histamine; antihistamine; erythropoietin; antifungals; antiparasitic agents; non-corticosteroid anti-inflammatory agents; anticoagulants; anesthetics; analgesics; oncology agents; cardiovascular drugs; vitamins and other nutritional supplements; hormones; glycoproteins; fibronectin; peptides including polypeptides and proteins; interferons; cartilage inducing factors; protease inhibitors; vasoconstrictors, vasodilators, demineralized bone or bone morphogenetic proteins; hormones; lipids; carbohydrates; proteoglycans; antiangiogenins; antigens; demineralised bone matrix (DBM); hyaluronic acid and salts and derivatives thereof, polysaccharides; cellulose compounds and derivatives thereof, antibodies; gene therapy reagents; genetically altered cells, stem cells; mesenchymal stem cells with transforming growth factor; cell growth factors; type II collagen; elastin; sulfated glycosaminoglycan (sGAG), glucosamine sulfate; pH modifiers; methylsulfonylmethane (MSM); osteogenic compounds; osteoconductive compounds; plasminogen; nucleotides; oligonucleotides; polynucleotides; polymers; osteogenic protein 1 (OP-1); Lim Mineralization Protein-1 (LMP-1); cartilage; oxygen-containing components; enzymes; melatonin; vitamins; and nutrients.

46. The kit of claim 43, wherein said biocompatible polymeric compound is a biocompatible degradable polymeric compound.

47. The kit of claim 43, wherein said biocompatible polymeric compound is a biocompatible non-degradable polymeric compound.
